(19) United States
(12) Patent Application Publication
(10) Pub. No.: US 2005/0085737 A1
(43) Pub. Date: Apr. 21, 2005

(54) Stethoscope with improved physical features

(76) Inventor: Kimothy C. Levia, Ile Bizard (CA)

Correspondence Address: Rodney L. Skoglund;Renner, Kenner, Greive, Bobak, Taylor & Weber, Fourth Floor, First National Tower, Akron, OH, 44308, US

(21) Appl. No.: 10/688,824

(22) Filed: Oct. 17, 2003

(51) Int. Cl. A61B 5/025

(52) U.S. Cl. 600/528

(57) ABSTRACT

The present invention generally relates to a mechanical technician's stethoscope for detecting, locating and diagnosing sounds generated by mechanical instruments. More particularly, this invention relates to a mechanical technician's stethoscope, including a telescoping sound-conducting shaft, and a light selectively shining along the telescoping line as defined by the telescoping sound-conducting shaft, wherein a handle portion of the stethoscope is configured to give the stethoscope a pistol-like design with functional benefits derived from such configuration.

### TECHNICAL FIELD

The present invention generally relates to a mechanical technician's stethoscope for detecting, locating and diagnosing sounds generated by mechanical instruments. More particularly, this invention relates to a mechanical technician's stethoscope, including a telescoping sound-conducting shaft, and a light selectively shining along the telescoping line as defined by the telescoping sound-conducting shaft, wherein a handle portion of the stethoscope is configured to give the stethoscope a pistol-like design with functional benefits derived from such configuration.

### BACKGROUND OF THE INVENTION

Engines and other types of machinery, for example, internal combustion engines, diesel engines, electric motors, bearings, generators, compressors, and pumps, generally include rotating and moving parts that generate distinctive acoustic signals and sounds. The acoustic signals and sounds emitted may be used to detect the existence, location and cause of abnormalities in the functioning of the mechanical element. For example, by analyzing acoustic signals from an engine, abnormalities such as piston knock, valve clatter, pressure leaks, bearing and bushing failure, broken or chipped gear teeth, and frictional abrasion might be identified and located for subsequent repair.

Because the sound emitted by a mechanical element yields valuable information as to whether or not the element is functioning properly, mechanics now commonly employ stethoscope-type devices for detecting, locating and diagnosing sounds generated by mechanical devices. The prior art generally provides an electronic stethoscope with a sound probe for detecting potential mechanical faults in a mechanical device. For example, an electronic stethoscope might be used to detect automotive engine sounds indicative of mechanical faults such as valve chatter, tappet noise, piston slap, gear and pump noises, and the like.

U.S. Pat. No. 5,445,026 discloses a sound diagnostic instrument for locating and discriminating automotive engine and industrial machinery abnormalities. The device therein includes a deformable sound probe arm that houses a microphone offset from an open end of the probe. This microphone is electrically interconnected with circuitry within the main housing of the device, and the circuitry serves to convert acoustic signals and sounds into electromagnetic signals that are monitored by preamplifier circuitry and decibel meters. The electromagnetic signals are also converted to secondary acoustic signals that may be listened to by the operator through an earphone headset that is electrically interconnected to the circuitry. While this device has been found to be useful, it should be noted that it focuses on providing a deformable sound probe of a fixed (although deformable) length. With this or a similar device, it may be difficult to access and acoustically diagnose a given mechanical part, especially if that part is fixed beyond reach and/or behind other components.

Thus, there exists a need in the art for a mechanical technician's stethoscope having a significantly adjustable sound probe or sound-conducting shaft. Additionally, there exists a need in the art for a mechanical technician's stethoscope having a light source to aid in ensuring that the proper mechanical part is being analyzed when the sound-conducting shaft is extended to a hard-to-reach component. Finally, a mechanical technician's stethoscope would benefit from, and there is a need for, a design that allows for easy and accurate placement of the sound-conducting shaft and selective operation of the stethoscope.

### SUMMARY OF THE INVENTION

In one embodiment, the present invention provides a stethoscope comprising a telescoping sound-conducting shaft having a fixed end opposite a distal end; a sound probe at said distal end of said telescoping sound-conducting shaft; and a housing retaining said fixed end of said telescoping sound-conducting shaft, such that said sound probe may be selectively positioned at different distances from said housing.

In another embodiment of this invention, a stethoscope is provided comprising a sound-conducting shaft having a fixed end and a distal end; a sound probe at said distal end of said sound-conducting shaft; a housing including a barrel portion that retains said fixed end of said sound-conducting shaft such that said sound-conducting shaft defines a substantially straight line from said fixed end to said distal end; and a sight light positioned in said barrel portion to shine along said substantially straight line defined by said sound-conducting shaft.

In yet another embodiment of this invention, a stethoscope is provided comprising a sound-conducting shaft having a fixed end and a distal end; a sound probe at said distal end of said sound-conducting shaft; and a housing including a barrel portion that retains said fixed end of said sound-conducting shaft such that said sound-conducting shaft defines a substantially straight line from said fixed end to said distal end, and a grip portion extending downwardly from said barrel portion at from about 90 to 150 degrees to said substantially straight line defined by said sound-conducting shaft.

In a most preferred embodiment, the sound-conducting shaft is a telescoping shaft, a sight light is provided to shine along the telescoping line of the sound-conducting shaft, and a grip portion of the housing extends at an angle from the telescoping line to provide the stethoscope with a very general pistol-like design. In this preferred configuration, the sight light shines along the length of the telescoping sound-conducting shaft much like a sight for a pistol aims down the barrel of the pistol. In keeping with this design, a trigger switch may be provided at the grip portion at a position for actuation by a hand gripping the grip portion, much like a trigger of a pistol is pulled. Thus, in the most preferred embodiment, a stethoscope is provided that is easy to manipulate with one hand, easy to direct toward a desired noise-producing element to be listened to, particularly as the sight light is provided to help illuminate an element that might be in the shadow of overlying elements and the telescoping sound-conducting shaft may be extended to a substantial length.

From the discussion above, it should be generally appreciated that, by “shine along” it is meant that the sight light shines along a line that is substantially parallel to the substantially straight line defined by the sound-conducting shaft, whether telescoping or non-telescoping. The important consideration is that the sight light should illuminate the distal end of the sound-conducting shaft so that it is easy to view where this distal end is being placed.

The focus of the present invention is provided in the embodiments generally disclosed above. Indeed, the stethoscope of this invention may be electronic or digital, as known in the art, and the particular internal components that serve to read the sound conducted through the sound-conducting shaft and transfer those sounds to earphones of the stethoscope are not material to the practice of this invention. Thus, herein, various functional elements of the stethoscope are defined as “functionally communicating” with other elements of the stethoscope, and “functionally communicating” is to be understood as indicating that the identified elements communicate in a manner that allows for the standard operation and functioning of a stethoscope, particularly a mechanical technician's stethoscope. For example, as will be seen below, a microphone is provided near the fixed end of the sound-conducting shaft and is disclosed as “functionally communicating” with the fixed end of the shaft, from which it is to be understood that the sounds ultimately transmitted through the shaft are picked up by the microphone for transmission to earphones and/or a digital recording medium.

### DESCRIPTION OF THE DRAWINGS

FIG. 1 is a side view of a preferred embodiment of a stethoscope according to this invention;

FIG. 2 is a side view as in FIG. 1, with internal components and telescoping sections shown in phantom; and

FIG. 3 is a side view of the stethoscope, showing the sound-conducting shaft at full extension.

### PREFERRED EMBODIMENT OF THE INVENTION

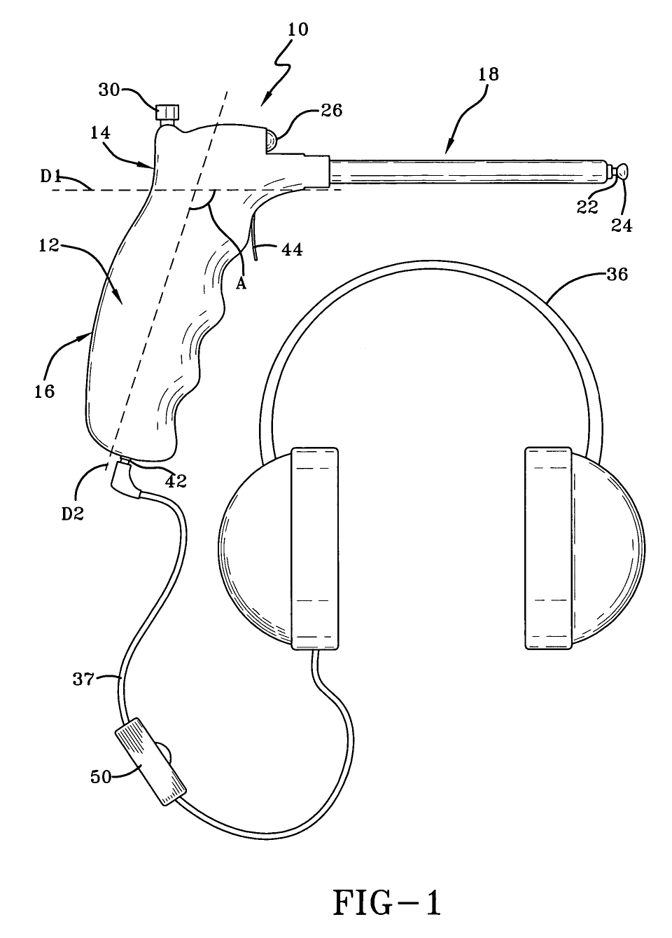

With reference to FIGS. 1-3, a preferred stethoscope according to this invention is shown and designated generally by the numeral 10. Stethoscope 10 includes a housing 12 that retains power sources and functional components that will be generally described below. It will be appreciated that housing 12, in this most preferred embodiment, is generally shaped in the likeness of a pistol, and, thus, a dashed line D1 has been included in FIG. 1 to segment housing 12 into a barrel portion 14 and a grip portion 16. A sound-conducting shaft 18 includes a fixed end 20 and distal end 22, and, the fixed end 20 is retained in barrel portion 14 of housing 12 such that the sound-conducting shaft 18 defines a substantially straight line from its fixed end 20 to its distal end 22. Notably, dashed line D1, which is used to distinguish between barrel portion 14 and grip portion 16 of housing 12, is substantially parallel to the substantially straight line defined between fixed end 20 and distal end 22, and this configuration is specifically chosen for the preferred embodiment inasmuch as barrel portion 14 and sound-conducting shaft 18 may be generally considered to be similar to the barrel of a pistol in the manner in which they relate to the design of stethoscope 10 as a whole.

Distal end 22 terminates at sound probe 24. Sound probe 24 provides a contact surface for placement against an element that is to be listened to, and is generally formed out of aluminum or other light metals and materials known in the art to satisfactorily conduct sound.

The pistol-like shape of stethoscope 10 makes it easy to employ inasmuch as the sound probe 24 at distal end 22 of sound-conducting shaft 18 may be positioned to contact a noise-producing element in much the same manner as a pistol is aimed. Positioning of the sound probe 24 would thus require only one hand, and the full range of motion of the operator's wrist could be put to use in an efficient manner to orient sound probe 24. A dashed line D2 is provided generally through the center line of grip portion 16 to show how grip portion 16 relates to barrel portion 14 and the line of extension of sound-conducting shaft 18. It will be recalled that dashed line D1, defining barrel portion 14, is drawn substantially parallel to the line defined by sound-conducting shaft 18, and dashed line D2, defining the extension of handle portion 16, crosses dashed line D1 at an angle A. In preferred embodiments of this invention, angle A ranges from about 90 degrees (i.e. handle portion 16 being perpendicular to barrel portion 14 and the line along which shaft 18 extends) to about 170 degrees. In other embodiments, this angle A may range from about 95 to 150 degrees, and, in yet other embodiments, from about 100 to 120.

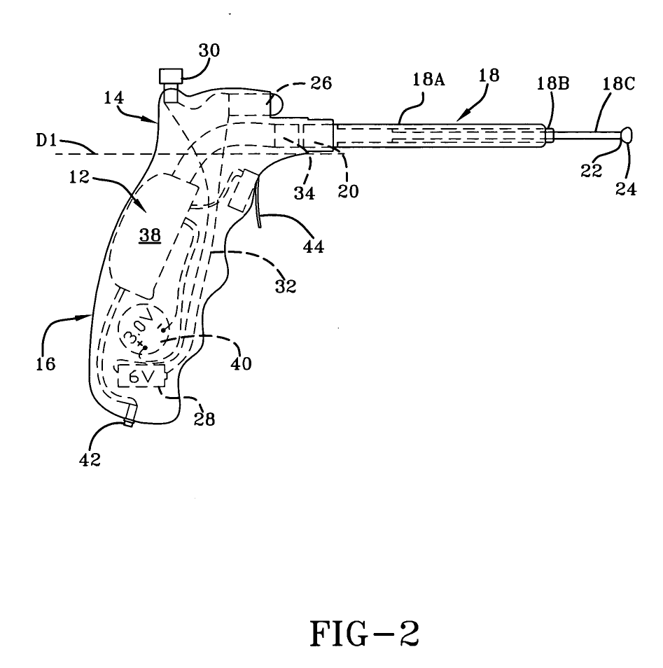

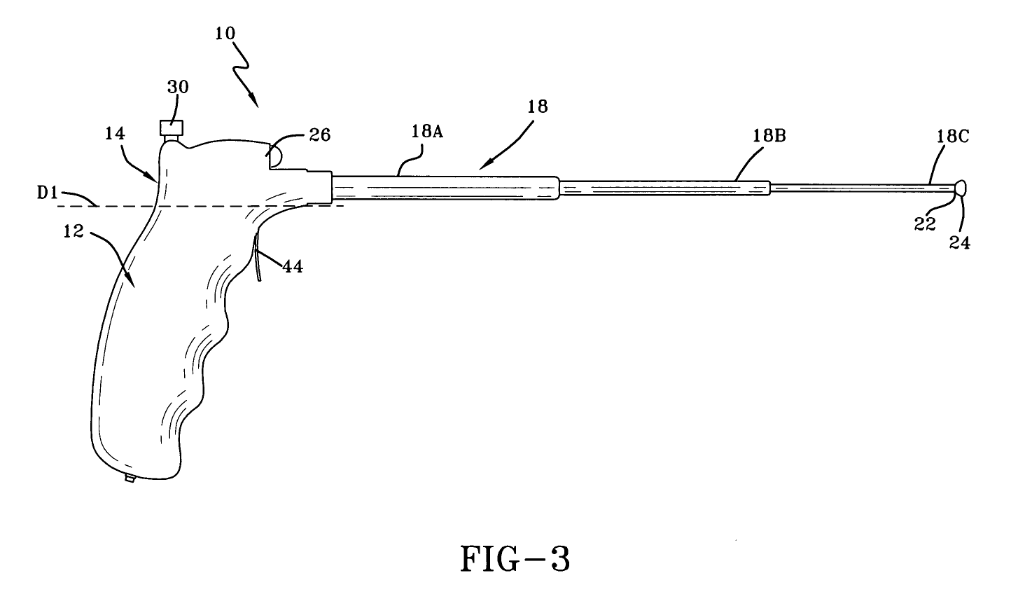

In particularly preferred embodiments, further utilitarian benefits are realized by having sound-conducting shaft 18 be telescoping, with a plurality of telescoping sections, such as first telescoping section 18A, second telescoping section 18B, and third telescoping section 18C, as particularly shown in FIGS. 2 and 3. By comparing FIGS. 1 and 3, it can be seen that a telescoping sound-conducting shaft 18 allows sound probe 24 to be selectively positioned at different distances from housing 12 (or fixed end 20), from the fully collapsed position of FIG. 1 to the fully extended position of FIG. 3.

It has already been mentioned that barrel portion 14 and sound-conducting shaft 18 are similar to the barrel of a pistol. In preferred embodiments, this aspect is further improved upon by providing a sight light 26, in barrel portion 14, to shine along the substantially straight line defined by sound-conducting shaft 18, whether telescoping or not. As mentioned above, sight light 26 “shines along” sound-conducting shaft 18 so that distal end 22 (and sound probe 24) may be viewed even when they are positioned in dark areas, for example, for listening to noises generated by a mechanical element that is positioned behind a number of other mechanical elements. A power source 28, such as a battery, connects sight light 26 and an on/off switch 30 by wires 32.

A microphone 34 functionally communicates with fixed end 20 of sound-conducting shaft 18, such that, when sound probe 24 is placed against a sound-producing element, the vibrations generated at this element are conducted through sound-conducting shaft 18 and picked up by microphone 34. Microphone 34 functionally communicates with earphones 36 so that the sounds picked up by microphone 34 may be heard. These sounds may be transmitted to earphones 36 either acoustically, electrically, or digitally, as known in the art. Here, wires 37 transmit the sound to earphones 36.

In the preferred embodiment herein, microphone 34 functionally communicates with a digital control unit 38, which, in any manner known in the art, converts sound data picked up at microphone 34 into digital sound data that may be recorded in digital media, if desired, and also transmitted to earphones 36. Control unit 38 is powered by a separate power source 40, such as a battery, and functionally communicates with earphones 36 through wires 37 and an appropriate jack 42 and wires 43.

The positioning of a trigger switch 44, so named because its positioning generally parallels the positioning of a trigger for a pistol, also facilitates use of stethoscope 10. This is clearly seen in the figures provided. When trigger switch 44 is actuated, control unit 38 and microphone 34 are activated to read and process sounds conducted through sound-conducting shaft 18, and these sounds are transmitted either to a recording device or earphones 36 or both in ways generally known in the art. In alternative embodiments, it might be considered desirable to position on/off switch 30 for sight light 26 at a position along grip portion 16 of housing 12 in order to allow for selective activation of sight light 26 by the same hand that selectively actuates the main sound reading/recording function of stethoscope 10. In one alternative embodiment, trigger switch 44 is also connected to sight light 26 such that sight light 26 is activated every time the reading/recording function is activated.

It may be desired to provide volume control for earphones 36, and, in a preferred embodiment, this is accomplished by providing a volume control unit 50 on the wires 37 leading to earphones 36. Such volume control units are generally known in the earphone arts, although it is believed that their use has not been explored in the stethoscope arts.

The stethoscope of this invention is used by first considering what length of sound-conducting shaft 18 is needed for reaching the sound-producing element to be listened to, and extending or contracting shaft 18 to the desired length. Thereafter, housing 12, particularly grip portion 16, is grasped and sound probe 24 is placed in contact with the sound-producing element. The stethoscope is gripped and manipulated much like a pistol, and sound probe 24 is positioned in a manner similar to aiming a pistol. If the sound-producing element is positioned in a dark or shadowed area, sight light 26 is preferable activated to help illuminate the positioning of sound probe 24. Once sound probe 24 is in contact with the desired element, the stethoscope is further manipulated like a pistol by pulling trigger switch 44 to activate the stethoscope so that sounds are transmitted through sound-conducting shaft 18 to microphone 34 and, ultimately, either a recording medium (particularly when the sound is converted to digital sound data) or earphones 36 or both.

In light of the foregoing, it should be apparent that the present invention provides a stethoscope that substantially advances the art. While only particularly preferred embodiments have been disclosed herein, this invention is not limited to or by any specific recitation of the elements of these embodiments and their interrelation. Notably, means for transmitting sound from a sound probe to earphones are generally known in the art, and any such means, as well as new means that might be provided in the future, may be incorporated into the present invention. The scope of this invention is limited only by the claims that follow.

1. A stethoscope comprising:
a telescoping sound-conducting shaft having a fixed end opposite a distal end;
a sound probe at said distal end of said telescoping sound-conducting shaft;
a housing retaining said fixed end of said telescoping sound-conducting shaft, such that said sound probe may be selectively positioned at different distances from said housing.

2. The stethoscope of claim 1, wherein said housing includes a barrel portion that retains said fixed end of said telescoping sound-conducting shaft to define a substantially straight telescoping line along which said sound probe may be selectively positioned, and a handle portion extending downwardly from said barrel portion at from about 90 to about 150 degrees to said telescoping line.

3. The stethoscope of claim 2, further comprising a trigger switch.

4. The stethoscope of claim 3, wherein said trigger switch is positioned at said stock portion for actuation by a hand gripping said handle portion.

5. The stethoscope of claim 4, further comprising a microphone functionally communicating with said fixed end of said telescoping sound-conducting shaft to read sound data conducted through said telescoping sound-conducting shaft.

6. The stethoscope of claim 5, wherein said trigger switch functionally communicates with said microphone to actuate said microphone to selectively read said sound data conducted through said telescoping sound-conducting shaft.

7. The stethoscope of claim 6, further comprising a digital control unit that functionally communicates with said microphone to receive sound data transmitted by said microphone and convert said sound data to digital sound data.

8. The stethoscope of claim 2, further comprising a sight light positioned in said barrel portion to shine a light along said telescoping line.

9. The stethoscope of claim 8, further comprising an on/off switch for said sight light.

10. A stethoscope comprising:
a sound-conducting shaft having a fixed end and a distal end;
a sound probe at said distal end of said sound-conducting shaft;
a housing including a barrel portion that retains said fixed end of said sound-conducting shaft such that said sound-conducting shaft defines a substantially straight line from said fixed end to said distal end; and
a sight light positioned in said barrel portion to shine along said substantially straight line defined by said sound-conducting shaft.

11. A stethoscope comprising:
a sound-conducting shaft having a fixed end and a distal end;
a sound probe at said distal end of said sound-conducting shaft;
a housing including:
a barrel portion that retains said fixed end of said sound-conducting shaft such that said sound-conducting shaft defines a substantially straight line from said fixed end to said distal end, and
a stock portion extending downwardly from said stock portion at from about 90 to 150 degrees to said substantially straight line defined by said sound-conducting shaft.
